(19) United States
(12) Patent Application Publication
(10) Pub. No.: US 2003/0113908 A1
(43) Pub. Date: Jun. 19, 2003

(54) Compost tea apparatus and methods

(76) Inventors: Leon R. Hussey III, Redmond, WA (US); Bill W. Gebhardt, Redmond, WA (US)

Correspondence Address: SEED INTELLECTUAL PROPERTY LAW GROUP PLLC, 701 FIFTH AVE, SUITE 6300, SEATTLE, WA, 98104-7092, US

(21) Appl. No.: 10/024,854

(22) Filed: Dec. 18, 2001

(51) Int. Cl. C05F 11/08; C12M 1/04

(52) U.S. Cl. 435/296.1; 435/290.2; 71/9

(57) ABSTRACT

The improved compost tea producing apparatus and methods of the present invention are directed to aeration means which substantially increases the count of beneficial organisms in liquid compost tea compositions. Such aeration apparatus of the present invention includes an air blower having an air provision capacity of at least approximately 350-500 ft/min. An air conduit is connected to the output port of the air blower, with such conduit having a generally vertical portion and such vertical portion having an effluent opening. An air diffuser tube is attached to the effluent opening of the vertical portion of the air conduit, with such air diffuser tube having a plurality of air bubbling apparatus as disposed along the length thereof for transmitting air into a volume of liquid compost tea.

### BACKGROUND OF THE INVENTION

1. Field of the Invention

This invention relates generally to the improvement of soil and plant health, and more particularly to improved apparatus and methods for producing a liquid soil nutrient composition known as “compost tea.”

2. Description of the Related Art

Since virtually time immemorial, it has been a goal of the developing agricultural practice and more recently agricultural science, to improve the soil, and most particularly and beneficially, by various organic means. To that end, a wide variety of organic slurries known as “compost teas” have been developed by a wide variety of various means. The composition known as Compost tea, as referred to herein contains a variety of living organisms which contribute to the beneficial properties of the soil and plants. Accordingly, compost tea is a form of organic fertilizer.

Compost tea has been found to have a multiplicity of benefits and advantages, including inter alia foliar feeding (i.e., feeding through the leaf structure), soil pest control, disease control, as well as soil revitalization. Indeed, compost tea has been shown to accomplish various of these advantages simultaneously, such as, for example, controlling fungus while providing nutrients to the plant. In that regard, the mechanism of operation of compost tea has been diversely defined, inasmuch as compost tea is a mixture of a wide variety of different nutrients, as well as organisms.

Despite the known benefits, of compost tea, many gardeners have failed to take advantage of these beneficial properties, due in material part to the expense and/or difficulty of producing compost tea. More specifically, various apparatus and/or methods have been proposed for producing compost tea. However these apparatuses and/or methods have generally been structured with elements for producing large commercial-batch apparatus, which have thus been largely unsuitable for the non-commercial market, including, for example, many hobbyists and gardeners.

Compost tea generally functions as a fertilizer, disease suppressor, and pest organism reducer, improver of soil structure with H₂O infiltration, O₂ oxygen diffusion, and increased soil capacity to retain water, hormone producer for improved plant growth, soil pollutant breakdown, functioning, as well as turning organic matter into humus which is available for plant root growth, and, finally, inoculating the soil with beneficial bacteria organisms. Such liquid compost tea compositions have beneficially confronted leaf spot, curly top, mildew, downy mildew, early or late blight, powdery mildew, damping off rust diseases, leaf diseases, wilts, rots, mosaic virus, bacterial blight, black rot, fusarium verticillium wilt, and anthracnose, to mention a few.

One drawback, however, of compost tea is that it generally must be used within three to four hours after brewing. However, many prior art compost tea producing systems have had an inherent “over-capacity” which has resulted in wasted ingredients, and hence increased costs.

In light of the above difficulties and disadvantages of prior art compost tea systems, it is a material object of some preferred embodiments of the present invention to alleviate these prior art concerns by providing improved structures and/or methods which will produce compost tea quickly and efficiently, and at a reduced cost to the consumer.

Several attempted solutions to the difficulties associated with producing compost tea compositions have been proposed. For example, one such proposed apparatus utilizes a nozzle design adapted from an automobile carburetion system to entrain oxygen into the liquid compost solution in conjunction with a pump to mix and discharge the liquid. Yet another proposed apparatus utilizes an electric motor attached to the mixing container per se to introduce air and to circulate continuously the liquid to provide oxygenation to the slurry volume. However, such nozzle-related and/or mixing-related devices and methods have unfortunately resulted in an increased cost of production of liquid compost tea, which disadvantage is materially alleviated by the improved functionality of the far simpler structure preferred embodiments of the present invention, which most beneficially uses only upwardly bubbling aeration means from a air diffuser element which is static.

### BRIEF SUMMARY OF THE INVENTION

The improved compost tea producing apparatus and methods of the present invention are directed to aeration means which substantially increases the count of beneficial organisms of liquid compost tea compositions. Such aeration apparatus of the present invention includes an air blower having an air provision capacity of at least approximately 350-500 ft/min. An air conduit is connected to the output port of the air blower, with such conduit having a generally vertical portion and such vertical portion having an effluent opening. An air diffuser tube is attached to the effluent opening of the vertical portion of the air conduit, with such air diffuser tube having a plurality of air bubbling apparatus as disposed along the length thereof for transmitting air into a volume of liquid compost tea.

The air diffusion tube in some preferred embodiments may comprise PVC or other polymeric pipe, or copper, stainless steel or other compositions, which are readily drillable for forming a plurality of air diffusion apertures therein, and which is also bendable into various shapes, preferably to match the cross-sectional shape of the container to be used for brewing the liquid compost tea composition. Thus, in some preferred embodiments, the diffuser tube is formed into a coil-shape in plan view for mating engagement with the circular-shaped bottom of a five-gallon container, for example.

In addition to such improved apparatus, the present invention also includes improved methods for brewing liquid compost tea compositions which include providing a quantity an aqueous-based liquid which is to be maintained at approximately 55°-80° C. Laboratory tested composts are added to the liquid in an approximate amount of 8 oz. of weight per approximately 5 gallons of water. Growth inducing nutrients, such as kelp and yeast, are also added to such liquid in the approximate amount of 3 oz. per 5 gallons of water. Air is then bubbled into the liquid/organism/nutrient slurry composition at approximately 350-500 feet/min. for 18 hours in order to achieve sufficient oxygenation of the liquid slurry composition to permit the beneficial organisms therein to multiply to an amount of approximately 5-500 times their original numbers.

### BRIEF DESCRIPTION OF THE SEVERAL VIEWS OF THE DRAWING(S)

Some preferred embodiments of the approved aeration apparatus of the present invention, and in which preferred methods thereof can be carried out, are depicted in the accompanying drawings, and in which:

FIG. 1 is a top view of the generally coil-shaped air diffusion tube, which, being generally circular in shape, is configured to matingly engage with the generally circular-shaped bottom surface of one preferred container;

FIG. 2 is a side view of the embodiment of FIG. 1, and also showing the vertical portion of the air conduit, which is to be connected at its top extremity to an air blower for providing air to the air conduit;

FIG. 3 is a perspective view of the improved aeration apparatus of FIGS. 1 and 2; and

FIG. 4 is a side view of the aeration apparatus of the present invention further depicting schematically an air blower apparatus disposed at the top portion of the air conduit thereof.

FIG. 5 is a perspective view of a different embodiment of the aeration apparatus of the present invention showing the air diffuser as a plate disposed near the bottom of the container such that air can bubble upwardly therefrom, with the air conduit in the form of a flexible tube, and the air blower disposed beside the container.

Other embodiments, elements, and features of the approved aeration apparatus and methods of the present invention will become apparent to those skilled in the art upon reviewing the following detailed description of preferred embodiments.

### DETAILED DESCRIPTION OF THE INVENTION

Aerated compost tea generally functions as a fertilizer, disease suppressor, and pest organism reducer, improver of soil structure with H₂O infiltration, O₂ oxygen diffusion, and increased soil capacity to retain water, hormone producer for improved plant growth, soil pollutant breakdown, functioning as well as turning organic matter into humus which is available for plant root growth, and, finally, inoculating soil with healthy beneficial organisms. Such liquid compost tea compositions have beneficially confronted leaf spot, curly top, mildew, downy mildew, early or late blight, powdery mildew, damping off rust diseases, leaf diseases, wilts, rots, mosaic virus, bacterial blight, black rot, fusarium verticillium wilt, and anthracnose, to mention a few.

One drawback, however, of brewed compost tea is that it generally must be used within three to four hours after completion of brewing. However, many prior art compost tea producing systems have had an inherent “over-capacity” which has resulted in wasted ingredients, and hence increased costs.

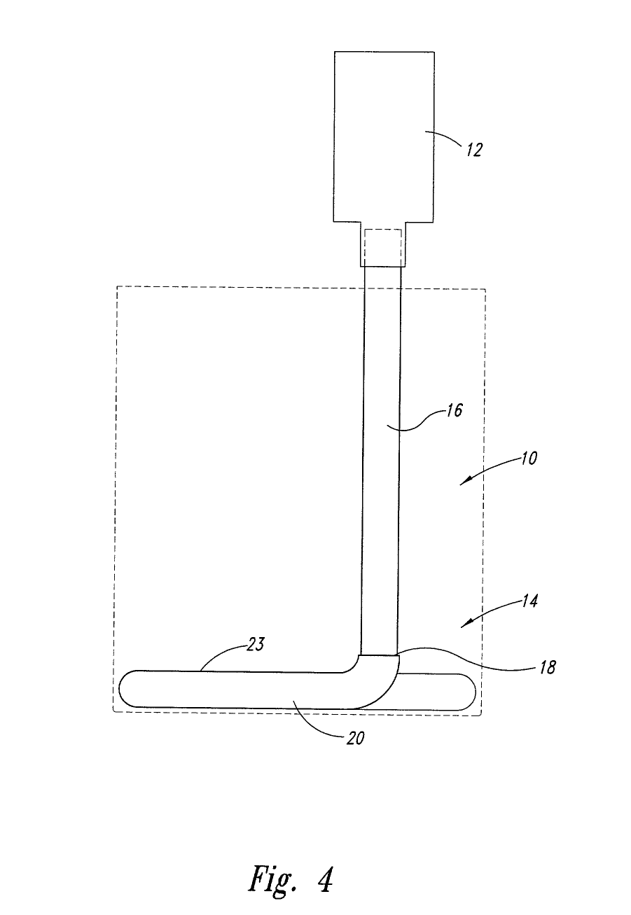

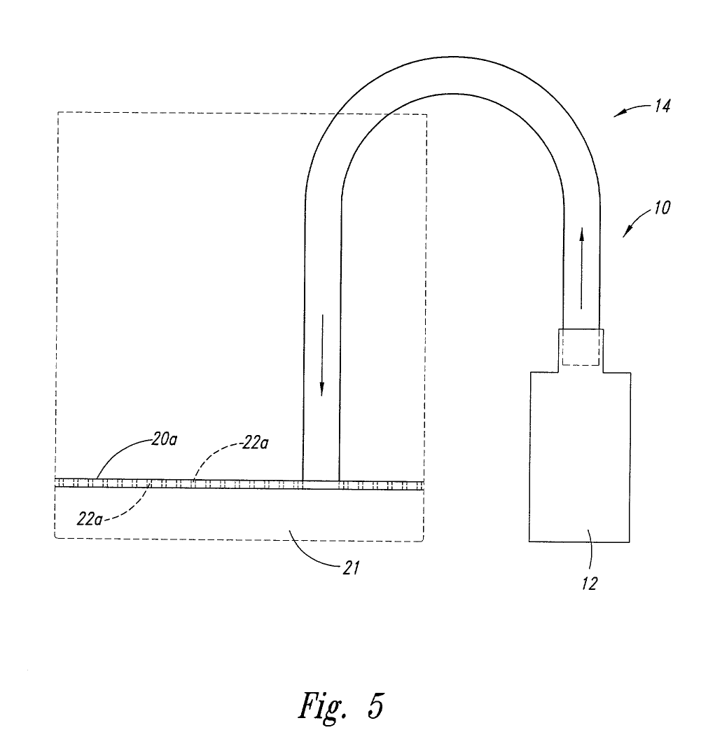

Referring now to the figures, the improved aeration apparatus of the present invention (generally 10) functions to increase the beneficial organism count of liquid compost tea compositions which are generally produced within a suitably shaped and sized container (not shown). An air blower apparatus 12, as generally shown in FIGS. 4 and 5, is provided and should have a capacity of at least approximately 350-500 ft/min. An air conduit 14 is connected to the output of the air blower 12 and has at least a generally vertical portion 16 thereof with the generally vertical portion 16 having an effluent opening 18 thereof.

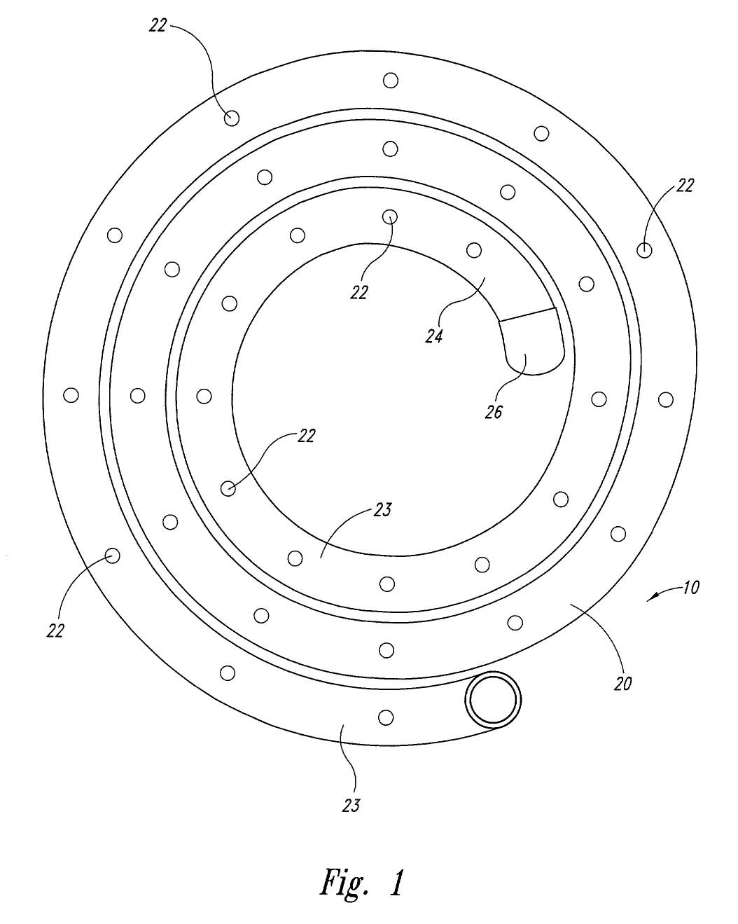

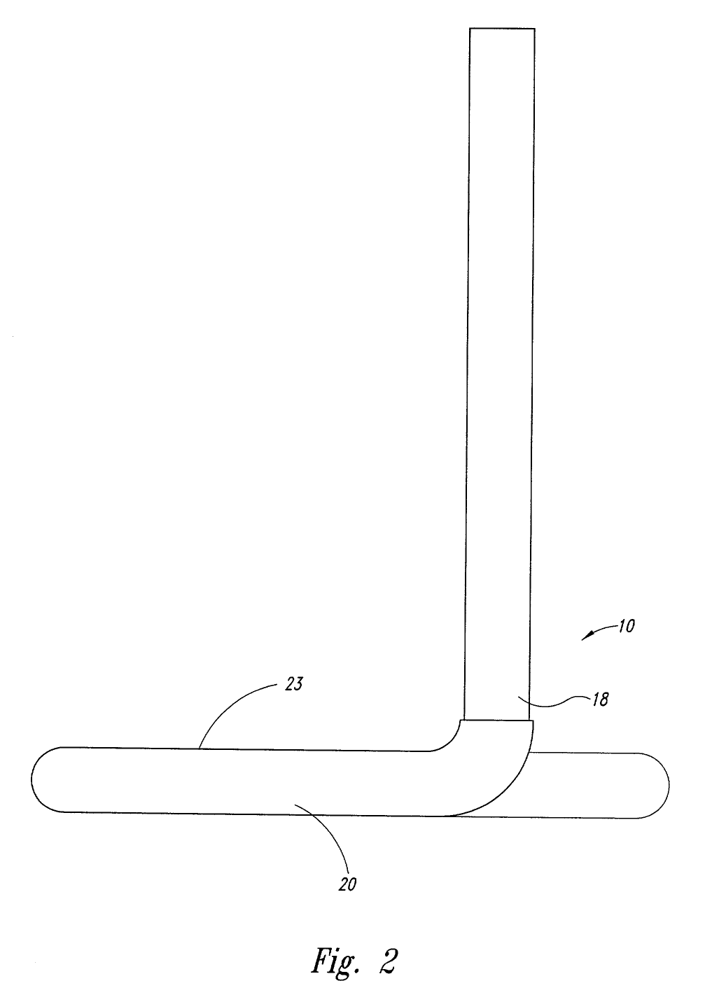

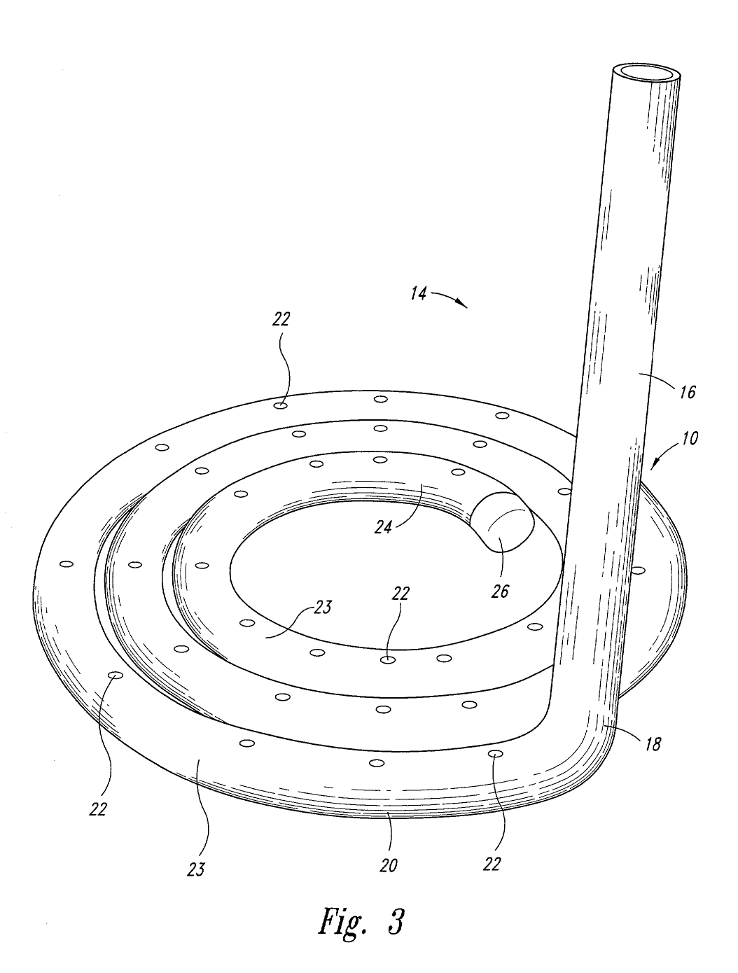

An air diffuser is in the form of a tube 20 in the embodiments of FIGS. 1-4 and in the form of a plate 20a in the embodiment of FIG. 5. Such air diffuser 20 is attached to the effluent opening 18 of vertical portion 16 of air conduit 14, as shown in FIGS. 2-4, for example. Vertical portion 16 and air diffuser tube 20 or air conduit 14 may alternatively be formed in separate pieces as shown in FIGS. 2 and 4, or in a unitary construction as shown in FIG. 3. The air diffuser tube 20 has a plurality of air bubbling apertures 22, as shown in FIGS. 1 and 3 which are disposed within upper surface 23 and along the length thereof for transmitting air therethrough into the liquid compost tea contained within the container (not shown) for aerating the liquid compost tea composition. Air diffuser plate 20a of FIG. 5 has similar air bubbling apertures 22a.

Air conduit 14 and its constituent parts may comprise in preferred embodiments PVC pipe, although other polymeric or other pipes such as copper or stainless steel may also be utilized. Such PVC pipe as shown in the embodiments hereof is approximately 0.5 inches in diameter, although other diameters may be utilized consistent with directing a sufficient volume and rate of air therethrough. Of course, in the embodiments of FIGS. 1-4 where the air blower is supported by the air conduit and disposed above the container, such air conduit must be substantially rigid, whereas in the embodiment of FIG. 5, the air conduit should most preferably be flexible to permit disposition of the air blower beside the container. For example, the apertures depicted in FIGS. 1 and 3 in the air diffusion tube are approximately 0.095 inches in diameter.

Such air diffuser 20 is capped at the distal end thereof 24 by a cap structure 26, and accordingly the air which is directed downwardly from the air blower element 12 into generally vertical portion 16 of air conduit 14 must exit through the apertures 22, 22 contained within upper surface 23 of the air diffuser tube 20. As indicated, supra, the air diffuser tube in the preferred embodiment set forth in FIGS. 1-4 hereof is formed into a coil shape, generally by bending in preferred embodiments, which generally conforms to the cross-sectional shape of the bottom of a preferred container, such as, for example, a five-gallon container. In the embodiments of FIGS. 1-4, the number of separate coils is to be determined by the volume of air which is disposed by the selected air pump to be blown through the selected number of apertures 22, 22.

FIG. 5 shows a further embodiment of the present invention. FIG. 5 is a perspective view of a different embodiment of the aeration apparatus 10 of the present invention showing the air diffuser as a plate 20a disposed near the bottom of the container (in dotted lines), such that air can bubble upwardly therefrom, and from air chamber 21, with the air conduit 14 in the form of a flexible tube, and the air blower 12 disposed beside the container.

In the methods of the present invention for brewing liquid compost tea, a suitable quantity of aqueous-based liquid is provided, which in some preferred embodiments is contained within a relatively small and generally non-commercial sized container, such as a five gallon bucket. The liquid compost tea composition should be maintained at least initially at approximately 55°-80° F. Next, in preferred embodiments, compost tea solids containing beneficial organisms and nutrients are added to the liquid compost tea generally in amount of 8 oz. per 5 gallons of water. Growth inducing nutrients are also added to the liquid composition. Thereafter, air is bubbled into the liquid slurry composition generally at a rate of 350-500 cubic feet per minute for a period of approximately 18 hours, such time selected in order to achieve a liquid compost tea composition having sufficient oxygenation to permit the beneficial organisms thereof to multiply to an amount of approximately 5-500 times the original numbers.

Thereafter, the brewed liquid tea composition is applied to a plant prior to substantial diminution of the multiplied amount of beneficial organisms contained therewithin. Generally speaking, such brewed liquid compost tea compositions must be applied within approximately 4 hours of their creation to achieve the maximum benefit.

As used herein the term “beneficial organisms” refers to bacteria, nematodes, protozoa, and fungi naturally occurring in a healthy soil. In general, in a well made compost, only beneficial organisms are present. The compost which contains such beneficial organism and which is to be added to the initial liquid for brewing generally comprises the brand name of “Ground Up”, from Ground Up Corp. of Seattle, Wash., which has a composition of straw, gypsum, poultry dust, rock dust, vegetable food waste, and clean paper/wood products. The nutrients which are added to the bacterial/liquid brew in some preferred embodiments are sold under the brand name “Compost Tea Catalyst” produced by Growing Solutions, Inc. of Eugene, Oreg. (www.growingsolutions.com”), and generally comprise the following nutrients: Certified organic cane juice crystals, soluble sea weed powder and yeast autolysate.

COMPOST TEA LAB RESULTS

| | | | | | | TOTAL | ACTIVE/ | ACTIVE/ | ACTIVE/ |
|---|---|---|---|---|---|---|---|---|---|
| | ACTIVE | TOTAL | ACTIVE | TOTAL | | FUNGAL/ | TOTAL | TOTAL | TOTAL |
| | BACT. | BACT. | FUNCAL | FUNGAL | HYPHAL | TOTAL BACT. | FUNGAL | FUNGAL | FUNGAL |
| SAMPLE | BIOMASS | BIOMASS | BIOMASS | BIOMASS | DIAMETER | BIOMASS | BIOMASS | BIOMASS | BIOMASS |
| Desired Range | 10-150 | 150-300 | 2-10+ | 2-20 | see chart | see chart | see chart | see chart | see chart |
| Example 1 | 39.9 | 149 | 1.98 | 3.86 | 2.5 | 0.026 | 0.512 | 0.268 | 0.049 |
| 6 cups | | | | | | | | | |
| com.3 oz.cat | | | | | | | | | |
| 24 hours | | | | | | | | | |
| Example 2 | 62 | 206 | 3.43 | 4.28 | 2.5 | 0.021 | 0.801 | 0.301 | 0.055 |
| 3ups | | | | | | | | | |
| comp.3 oz.cat | | | | | | | | | |
| 12 hours | | | | | | | | | |
| Example 3 | 31.2 | 150 | 0.45 | 0.55 | 2.5 | 0.004 | 0.807 | 0.209 | 0.014 |
| 1c. comp.3 | | | | | | | | | |
| oz.cat | | | | | | | | | |
| 12 hours | | | | | | | | | |
| Example 4 | 46.3 | 180 | 0.34 | 0.78 | 2.5 | 0.004 | 0.43 | 0.026 | 0.007 |
| 2c. comp.3 | | | | | | | | | |
| oz.cat | | | | | | | | | |
| 12 hours | | | | | | | | | |
| Example 5 | 14.9 | 797 | 0.0 | 69 | 2.5 | 0.52 | 0.0 | 0.02 0.0 | |

### EXAMPLES OF THE PRESENT INVENTION

Certain preferred apparatus and methods of the present invention were compared against a prior art apparatus. Specifically, the prior art was compared against four separate utilizations of the invention hereof with the beneficial results that active bacteriological biomass was substantially increased over the prior art. Active fungal bio-mass was substantially larger than in the prior art. Active/total bacterial biomass was substantially larger than the prior art, and active fungal/active bacteriological biomass was also substantially larger than in the prior art.

### Examples 1-4

In the applicant's Examples 1-4, the Apparatus of the present invention was run for the various times as indicated, utilizing different amounts of compost composition, and different amounts of catalyst, as indicated.

### Example 5

In Example 5, the prior art “In Harmony” apparatus produced by In Harmony of Woodinville, Wash. utilized a similar compost and catalyst composition.

### Example 6

The following techniques were utilized in formulating Examples 1-5, above.

Biomass of active (metabolizing) bacteria and fungal hyphae by staining with FDA and direct counts.

Biomass of fungal hyphae by direct count using agar film method.

Total bacterial biomass by direct count after staining with FITC.

Protozoa numbers estimated using Most Probable Number Tehnique.

### Example 7

The following parameters and/or definitions were utilized in carrying out Examples 1-6, supra.

FDA=Fluorescein diacetate, FITC=Fluorescein isothiocyanate, both are stains used to make bacteria and fungal hyphae visible under UV light.

The agar film method is a technique of suspending a soil solution in agar on a microscope slide. This creates a 3-dimentional space within which organisms are counted.

Most Probable Number is a statistical method of estimating organism numbers based in presence or absence in increasingly dilute solutions. A tissue culture plate to incubate protozoa on soil extract agar is used. Four replicates of each dilution from 1:10 to 1:1,000,000 are checked for the presence or absence of protozoa. Amoebae were present in all four reps of the 1:1-, 1:100 and 1:1000, and in two reps of the 1:10,000 dilution, but none were found in the higher dilutions. An MPN table shows that there are 13,863 amoebae present in 1 gram of fresh soil. See, Elaine Ingham, “Soil Protozoa” chapter (specifically p. 126), in David Sylyia, Principles and Applications of Soil Microbiology.

From the foregoing it will be appreciated that, although specific embodiments of the invention have been described herein for purposes of illustration, various modifications may be made without deviating from the spirit and scope of the invention. Accordingly, the invention is not limited except as by the appended claims and the equivalents thereof.

What is claimed is:

1. An aeration apparatus for increasing the beneficial count of beneficial organisms in liquid compost tea compositions disposable within a container, said apparatus comprising:
an air blower having an air provision capacity of at least approximately 350-500 ft/min;
an air conduit connected to the output of said air blower and having at least a generally vertical portion thereof, said generally vertical portion having an effluent opening thereof; and
an air diffuser attached to the effluent opening of said vertical portion of said air conduit, said air diffuser having a plurality of air bubbling apertures disposed within a surface thereof for transmittal of air therethrough into the liquid compost tea contained within said container for aeration of the liquid compost tea composition.

2. The aeration apparatus of claim 1 wherein the air diffuser comprises a tube.

3. The aeration apparatus of claim 1 wherein the air diffuser comprises a plate disposed within the container near the bottom thereof to create an air chamber for receipt of air from said air conduit.

4. The aeration apparatus of claim 1 wherein said air blower is disposed generally vertically above the container.

5. The aeration apparatus of claim 1 wherein said air blower is disposed beside the container.

6. The aeration apparatus of claim 1 wherein said air conduit is formed from a length of polymeric tubing.

7. The aeration apparatus of claim 5 wherein said air conduit is formed from flexible tubing.

8. The aeration apparatus of claim 1 wherein said air conduit comprises substantially rigid tubing.

9. The aeration apparatus of claim 1 wherein said air diffuser is formed from a length of substantially rigid PVC tubing.

10. The aeration apparatus of claim 1 wherein said air diffuser comprises polymeric pipe.

11. The aeration apparatus of claim 1 wherein said air conduit includes a substantially straight section of tubing.

12. The aeration apparatus of claim 1 wherein said air conduit and said air diffuser are is unitarily formed.

13. The aeration apparatus of claim 11 wherein said straight section of tubing is of strength sufficient to alone support said air blower above the container.

14. The aeration apparatus of claim 2 wherein said air diffuser is generally coil-shaped in plan view.

15. A method for brewing liquid compost tea comprising:
providing a quantity of aqueous-based liquid, said liquid maintained at approximately 55°-80° F.;
adding compost tea solids containing beneficial organisms to said liquid in an approximate amount of 8 oz. per 5 gallons of water;
adding growth inducting nutrients for said bacteria to said liquid; and
bubbling air into said liquid at a rate of approximately 350-500 ft/minute for approximately 18 hours to brew a liquid compost tea achieve sufficient oxygenation of said liquid to permit said bacteria to multiply to an amount of approximately 5-500 times their original number.

16. The method of claim 15 further comprising the step of applying said brewed liquid compost teat to a plant prior to substantial dimunition of said multiplied amount of beneficial organisms contained therewithin.
